(19) United States
(12) Patent Application Publication
(10) Pub. No.: US 2013/0165964 A1
(43) Pub. Date: Jun. 27, 2013

(54) ACTIVE PRESSURE CONTROL FOR VASCULAR DISEASE STATES

(71) Applicants: Karl Vollmers, Minneapolis, MN (US); Christopher Scorzelli, St. Paul, MN (US); Eric F. Little, Shakopee, MN (US); John Scandurra, St. Paul, MN (US)

(72) Inventors: Karl Vollmers, Minneapolis, MN (US); Christopher Scorzelli, St. Paul, MN (US); Eric F. Little, Shakopee, MN (US); John Scandurra, St. Paul, MN (US)

(73) Assignee: Regents of the University of Minnesota, St. Paul, MN (US)

(21) Appl. No.: 13/820,035

(22) PCT Filed: Sep. 21, 2011
(86) PCT No.: PCT/US11/52603

§ 371 Date: Feb. 28, 2013

Related U.S. Application Data
(60) Provisional application No. 61/384,906, filed on Sep. 21, 2010.

(51) Int. Cl. A61B 17/12

(52) U.S. Cl. CPC A61B 17/12
USPC 606/201

(57) ABSTRACT

A system includes a sensor, a controller and an actuator. The sensor is configured to provide a signal corresponding to pressure in a vascular system. The controller is configured to receive the signal and generate an output. The actuator is coupled to the controller and configured to modulate an element of the vascular system based on the pressure.

### CROSS REFERENCE TO RELATED APPLICATIONS

This application claims priority to U.S. Provisional Application Ser. No. 61/384,906, the entirety of which is hereby incorporated by reference.

### FIELD OF THE INVENTION

The invention relates generally to a system and method for active pressure control for vascular disease states, and more particularly to a system that reduces pressure in the vascular system.

### BACKGROUND OF THE RELATED ART

Pulmonary Hypertension (PH) is a condition characterized by elevated blood pressure in the pulmonary circulation. It can be caused by multiple diseases and if not controlled, leads to right heart failure and death. Depending on the form of the disease, afflicted individuals can have a poor quality of life and prognosis. According to one authority, median survival time for untreated idiopathic pulmonary arterial hypertension in 2002 was 2.8 years. PH is defined as a mean blood pressure in the pulmonary artery greater than 25 mmHg at rest.

A healthy artery is an elastic vascular structure that can deform when acted on by mechanical forces. With some diseases, such as arteriosclerosis and hypertension, an artery becomes less compliant than normal. Low compliance results in a relatively high pulsatile pressure in the artery for a given stroke volume. The reduction of compliance also alters the wave propagation velocity so that reflected waves contribute significantly to the pulse wave amplitude. The high pulsatile arterial pressure, in turn, causes high peak ventricular wall stress and energy expenditure. Over time, this increased cardiac burden can lead to heart failure, and ultimately, death.

Therefore, there is a need for new and improved devices for treating hypertension of the systemic or pulmonary circulations.

### BRIEF SUMMARY OF THE INVENTION

The present invention fulfills this need by providing novel systems and methods for treating hypertension of the systemic or pulmonary circulations.

In one aspect of the invention an adaptive device cancels or minimizes pulsatile pressure waves in the vascular system. The adaptive device includes at least one sensor, a processor and at least one actuator. The sensor or sensors detect pressure waves and convert it into an electrical signal. The processor receives an input signal from the sensor or sensors and generates an output signal. The actuator or actuators receive the output signal and produce a pressure wave which, when combined with the original wave, tends to minimize the wave amplitude.

These and other aspects of the present system, devices and methods are set forth in the Figures, Detailed Description and Claims.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A and 1B illustrate pressure traces from a normal person and from a patient with pulmonary hypertension showing the relative contribution of forward and reflected waves.

FIG. 2 illustrates an active compliant array for reducing pulse wave amplitude.

FIG. 3 illustrates an active compliant array for reducing pulse wave amplitude with actuators surrounding a vessel.

FIG. 4 illustrates an active extravascular cuff for minimizing pulse wave amplitude.

FIG. 5 illustrates an active wave cancellation system utilizing electromechanical actuators for minimizing pulse wave amplitude.

FIG. 6 illustrates an active wave cancellation system using vascular smooth muscle actuators.

### DETAILED DESCRIPTION OF THE INVENTION

Afterload is caused by the dynamic interplay between steady state resistance, dynamic stiffness and wave reflections. In Pulmonary Arterial Hypertension all three of these are altered to increase the workload on the heart.

Compliance is a measure of the ability of an elastic body to accommodate deformation. When considering a closed volume, compliance is defined as the ratio of the change of internal volume to the change in internal pressure. Mathematically, compliance can be expressed as C=ΔV/ΔP and is the multiplicative inverse (or reciprocal) of elastance and inversely proportional to stiffness.

The right and left ventricles of the heart pump blood into the pulmonary artery and aorta respectively. As the heart undergoes systole and diastole, pulsatile flow is generated such that localized periodic pressure rises and falls about the mean arterial pressure. A time response of blood pressure at a particular location along the artery exhibits a periodic variation of pressure levels about the mean that is correlated with systole and diastole.

In pulmonary hypertension (PH), the pulmonary vascular resistance (PVR), or resistance to mean flow, is increased. This can be due to disease in the distal portion of the pulmonary arterial vasculature. This leads to an increase in the mean or steady state pulmonary blood pressure. Historically, pulmonary hypertension researchers and physicians focused their efforts on this mean pulmonary pressure. But some research indicates that high mean pressure is only part of the problem. Increased pulsatile pressure, or the maximum pressure generated in the proximal pulmonary artery (PA) with each heartbeat, may be at least as important as increased mean pressure.

In PH, the pulmonary artery walls are abnormally stiff and noncompliant. With stiff vessel walls, each stroke volume results in a large increase in pulsatile pressure in the pulmonary artery. The right ventricle must work harder in order to sustain life, as demonstrated by measured increases in mechanical work, oxygen consumption and wall stress. Chronically, this increased workload triggers a complex sequence of events leading right heart failure. Decreased compliance of the PA is associated with increased adult mortality and poorer pediatric outcomes.

With each heartbeat, a localized transient increase in blood pressure occurs in the proximal pulmonary artery. This local pressure spike, or pulse, generates a wave that travels down the pulmonary vasculature at the phase velocity of sound. This is called pulse propagation velocity (PPV). The PPV is estimated by the Moens-Korteweg equation, which takes into account the PA physical characteristics such as compliance, wall thickness and diameter.

When a pulse wave encounters a physical discontinuity, such as the abrupt reduction of diameter in arterioles, a wave continues anterograde (forward, away from the heart) and a wave reflects and travels retrograde (towards the heart). In a normal, healthy individual, the PPV is such that the reflected wave reaches the heart during diastole (not during ejection of blood into the PA). In PH however, the PA wall thickness, diameter and compliance are altered such that PPV is increased. These physical changes can increase the PPV by as much as 30%, altering the timing of the reflected waves reaching the heart. In PH, the major reflected waves can arrive at the heart during ejection, significantly adding to the load the right ventricle must overcome to pump blood.

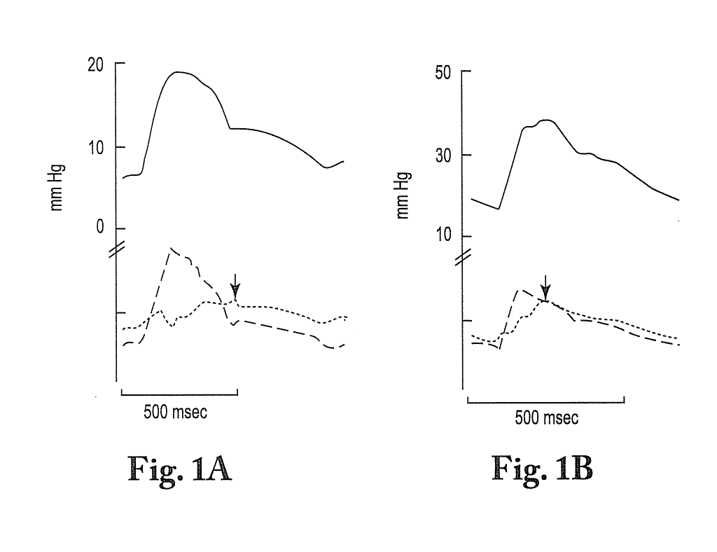

FIG. 1A illustrates pulse traces from a normal healthy person. FIG. 1B illustrates pulse traces from a patient with pulmonary hypertension patient. The top curve in each figure depict pulmonary pressure traces measured by right heart catheterization. The lower curve in each figure depicts the relative contribution from anterograde (dashed) and reflected (dotted) waves. The arrows define the peak contribution from reflected waves.

As shown in FIG. 1A, in the normal, healthy person, the reflected wave is relatively low amplitude and its peak occurs after ejection. In contrast, in the PH patient (FIG. 1B), the reflected wave is large and its peak occurs during ejection. Thus in PH, the reflected waves contribute significantly to afterload. The reflected wave contributes 31-38% of the pulsatile load according to one source.

As used in this specification, the following terms have the following meanings.

Sensor: A sensor is a transduction device used to monitor a physiological quantity such as displacement, pressure, velocity, acceleration, temperature, position, flow, magnetic or electrical activity. Sensors communicate transduced signals to other devices through various means including direct electrical connection, wireless communication, inductive and magnetic coupling. Sensors can be located upstream or the downstream of the device, co-located with the device, inside or outside the vasculature or both, or in or on the wall of the vasculature. Examples of sensors include, but are not limited to, microphones, hyprophones, accelerometers, antennas, and the like.

Control system: A control system is a computer-based processing system that transforms a single input or multi input signal to a single output or multi-output signal that is correlated with the input signal. Transform processing events that occur within the control system can include, but are not limited to, measurement and conditioning of sensor transduction signals, processing of sensor transduction signals with a mathematical algorithm and generation of output signals suitable for use by devices external to the control system. Mathematical algorithms may be customized depending on the particular application or adaptations of available algorithms such as those described in “Active Noise Control Systems: Algorithms and DSP Implementations,” by Kuo and Morgan (ISBN 0471134244).

Actuator system: An actuator system is a device that is capable of transforming a signal into a physical property such as force, displacement or pressure. An actuator system may typically consist of an actuator, a system controller and a power source.

Examples of actuators include piston pumps, bladder pumps, diaphragm pumps, vacuum pumps, speakers, shakers, piezoelectric sound and vibration sources, electromechanical sound and vibration sources, underwater sound sources and muscles. Examples of power sources include batteries, generators, AC power, thermal power, inductive power.

Anchor: An example of the present subject matter can be held in place, or anchored, by various structures. The present subject matter is anchored to reduce the risk presented by an embolized structure. For example, a device can be anchored by a suture, a stent, a friction fit, expansion to fill a hollow or vascular space, a hook mechanism, vascular endothelial in-growth, a barb mechanism, a rivet, compression exerted by adjacent tissue, glue, a tether to a body part, or a magnet.

Delivery: An example of the present subject matter can be delivered to the installation site by various procedures, including a surgical procedure or a percutaneous procedure. For example, general surgery, percutaneous transcatheter surgery, thorascopic surgery, and intra or extra vascular placement can be used. A minimally invasive surgical procedure can be used to install a device. A percutaneous installation procedure can include using a needle, an introducer guide wire, an introducer sheath, and a catheter. The catheter can also be used to move, adjust, activate, inflate or pressurize the device after installation. Such methods and tools can also be used for device removal or to reposition a device.

Fabrication: In one example, the device is fabricated of a material that is biocompatible. In addition, one example includes a biologically absorbable material. Other materials can also be used. For example, a material that assists in or impedes the growth of endothelial cells on a surface can be used for various components. In one example, a component is fabricated of a material having a smooth, low friction surface that facilitates implantation or removal. The device may be fabricated of a material that prevents or minimizes thrombosis, coagulation, platelet activation, or environmental degradation.

Device fabrication can include manufacturing a balloon. In addition, molded or formed materials, such as sheet goods, can be used in the fabrication of such a device. A fatigue resistant polymer having sufficient flexibility can be used for a membrane. In one example, a membrane is fabricated using a sputter-coating (diffusion layer) to limit gas pass-through.

The device may be constructed such that its mechanical properties are one of a spring-mass-damper with low resonance frequencies so that it may efficiently absorb oscillatory energy below 10 Hz. Consider one example in which the present subject matter is configured for use on an artery of a vascular system.

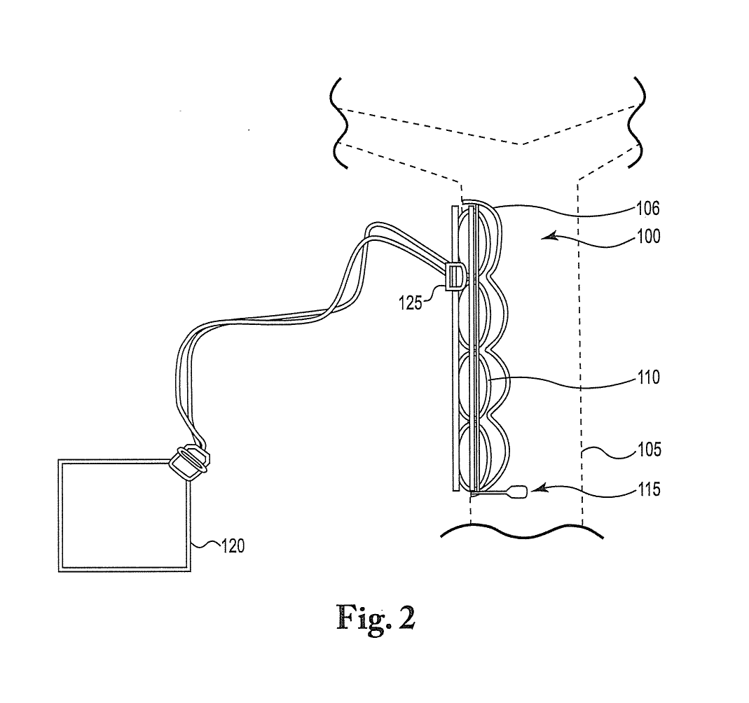

Referring now to FIG. 2, device 100 includes a compliant body 110 anchored to a vessel 105 on the interior or exterior side of the vessel. Sensor 115 is located upstream of the compliant body 110 and is arranged to sense incident pressure waves within the vessel 105 before the pressure waves reach compliant body 110.

Referring to FIG. 2, sensor 115 is located within vessel 105 and is in communication with control system 120 that processes the sensor signal and generates an output signal for an actuator 125 which in this case generates pressure in a fluid. Pressure waves created by the actuator are communicated to the compliant body 110. Sensor 115 may be located upstream and/or downstream of the compliant body, which may comprise a membrane, and inside and/or outside the vessel.

In operation, the sensor 115 is optimally positioned to sense pressure waves in a vessel 105. The compliant body 110 is anchored to the vessel 105 in a manner that causes the vessel wall 106 to dimple or protrude into the lumen defined by vessel 105 or anchored within the vessel 105 such that the volume of the compliant body 110 reduces the blood volume of the vessel 105 Upon sensing a pressure variation the control system 120 generates an output signal that causes the actuator 125 to vary pressure within the compliant body 110. The variation in pressure causes the compliant body 110 to deflate creating additional volume within the vessel 105 and thus, an apparent increase in vascular compliance. This action has the effect of slowing the incident and reflected pressure waves from a heartbeat such that the reflected wave is delayed and arrives back at the right atrium some time after systole reducing the afterload effect of the reflected wave. It also directly reduces the amplitude of pulse waves.

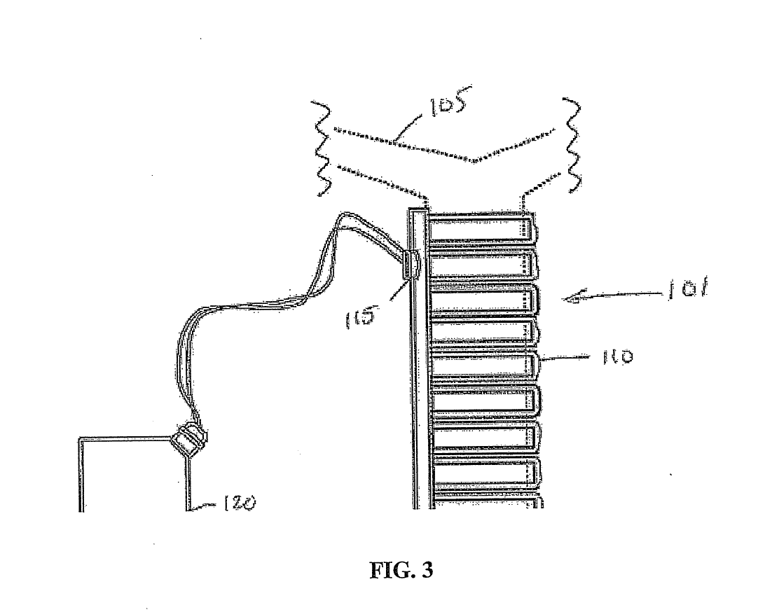

FIG. 3 illustrates another aspect of the invention in which device 101 is similar to that in FIG. 2 but with the compliant body surrounding the entire vessel.

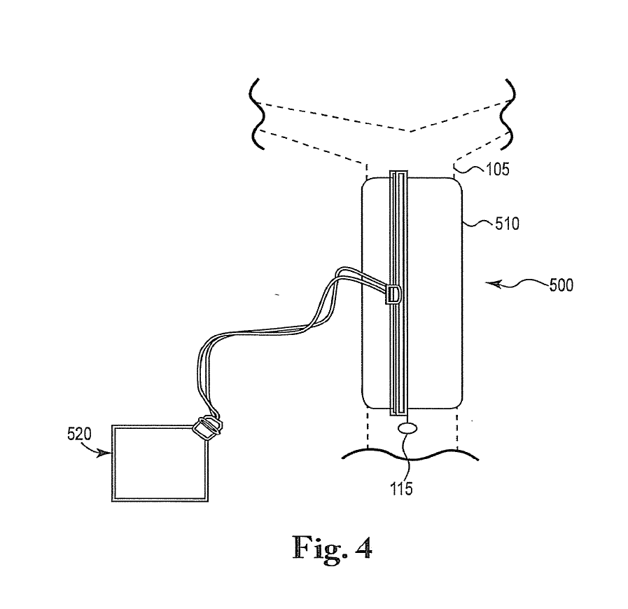

Referring now to FIG. 4 one aspect of the invention is depicted. Device 500 includes an actuator system 510 anchored to the exterior of a vessel 105. A sensor 115 is located upstream of the actuator 510 and is arranged to sense incident pressure waves within the vessel 105 before they reach the actuator system 510. FIG. 4 depicts sensor 115 as being located within the vessel 105. Those of skill in the art will appreciate, however, that sensor 115 may be located outside the vessel 105, upstream or downstream of the actuator 510. There may also be a plurality of sensors at a plurality of locations. Sensor 115 communicates to a control system 520 that processes the sensor signal and generates an output signal for the actuator system 510. Displacement variations or waves created by the actuator system are communicated to the vessel 105.

In operation, the sensor 115 is located in a position to sense pressure waves in the vessel 105. The actuator system 510 is anchored to the vessel 105 in a manner that causes the vessel wall 106 to dimple or protrude into the vessel lumen 105. Upon sensing a pressure greater than mean pulmonary artery pressure, the control system 520 generates an output signal that cause the actuator system 510 to displace from its null position. The displacement causes the vessel 105 to un-dimple creating additional volume within the vessel 105 and thus, an apparent increase in vascular compliance. This action has the effect of decreasing pulse pressure and slowing the incident and reflected pressure waves from a heartbeat such that the reflected wave is delayed and arrives back at the right atrium some time after systole reducing the afterload effect of the reflected wave. As sensor 115 senses a pressure less than mean pulmonary artery pressure, the control system 520 causes the actuator system 510 to return the vessel 105 to the originally dimpled position. This action has the effect of decreasing cross-sectional area in preparation for the next pressure cycle. The device can be arranged such that sensor is down stream to the actuator. The system can be configured to minimize the anterograde and/or reflected waves.

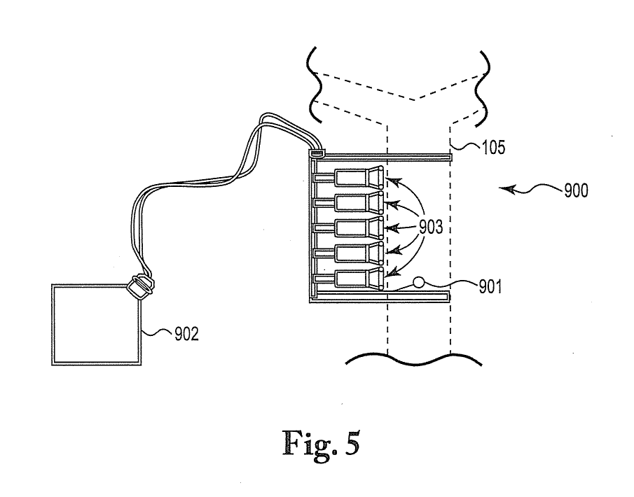

FIG. 5 illustrates device 900 according to one aspect of the invention. Device 900 includes one or more sensors (901), a processor (902) and one or more actuators (903). FIG. 5 depicts sensor 901 located within the vessel 105 for purposes of illustration only. However, all or parts or the system can be located inside or outside or adjacent to the inner or outer wall of vessel 105. Sensor 901 communicates to a control system 902 that processes the sensor signal and generates an output signal for the actuator system 903. Pressure waves created by the actuator system are communicated to the vessel 105. In operation, the sensor is positioned to sense pressure waves in the vasculature of interest. Sensor 901 may detect the anterograde or reflected waves. Processor 902 analyzes the signals generated by sensor 901 and generate an output signal based on adaptive cancellation algorithms. The actuator 903 will produce a pressure change in the vessel 105 such that the summation of the original wave and the system-generated wave will tend to minimize the original wave at a location of choice. This location can be chosen by tuning the system (adjusting the delay time between the input signal and output signal. Possible locations for the minimization of the original wave include important organs such as the heart, kidneys, brain, liver or locations of vessel defects or aneurisms. This will decrease pressure wave at the location of choice and decrease the effects of pressure on the important organ. In the situation that the important organ of choice is the heart, this will have the therapeutic effect of reducing the mechanical workload on the heart

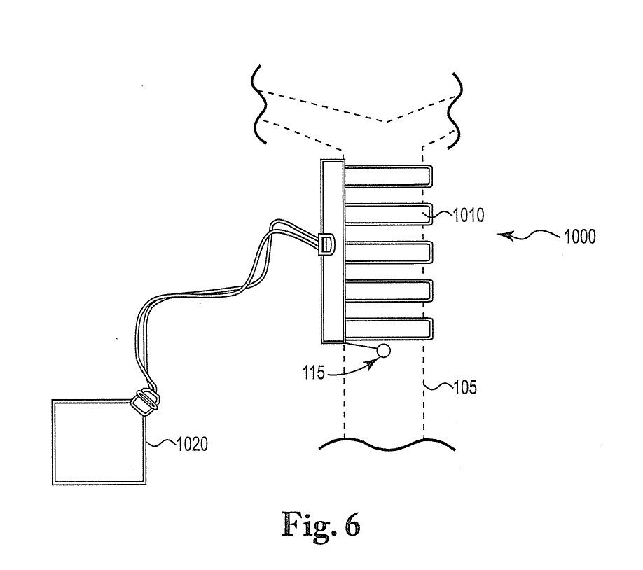

FIG. 6 illustrates device 1000 according to another aspect of the invention. Device 1000 includes an electrode 1010 implanted into the smooth muscle and anchored to a vessel 105. A sensor 115 is located upstream of the electrode 1010 and is arranged to sense incident pressure waves within the vessel 105 before the pressure waves reach electrode 1010. FIG. 6 depicts sensor 115 located within the vessel 105. Those of skill in the art will appreciate, however, that sensor 115 may be positioned in any location where incident pressure variation within the vessel 105 may be detected. Sensor 115 communicates to a control system 1020 that processes the sensor signal and generates an output signal for the electrode 1010. The electrodes stimulate the smooth muscle in the wall of the vessel to contract in a controlled manner.

In operation, device 1000 operates in a similar manner as device 900, but the actuator works by stimulating smooth muscle contraction in the wall or vessel 105.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code can be tangibly stored on one or more volatile or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed:

1. A system comprising:
at least one sensor configured to sense an initial pressure within a vessel and generate a signal corresponding to said initial pressure;
a controller configured to receive said signal and generate an output signal;
an actuator including means to alter said pressure in said vessel in response to said output signal such that the combination of the initial pressure and the altered pressure serve to cancel each other thereby modulating an after load effect.

2. The system of claim 1 wherein the compliant body is structured to be coupled to said vessel to cause a wall of said vessel to protrude into a lumen of said vessel.

3. The system of claim 1 further wherein the amplitude of pulse waves is reduced.

4. The system of claim 1 wherein said compliant body circumferentially surrounds said vessel.

5. The system of claim 1 wherein said sensor is positioned upstream of said compliant body.

6. The system of claim 1 further comprising at least one actuator coupled to said controller and configured to adaptively provide a modulated pulse wave to the vascular system thereby reducing pressure in a vasculature system.

7. The system of claim 6 wherein said at least one actuator is configured to produce a pressure change in said vessel such that the summation of an initial pressure wave and the modulated pulse wave minimizes the initial pressure wave.

8. The system of claim 1 wherein said controller outputs a signal to minimize the pressure wave at desired location.

9. The system of claim 1 further comprising an implantable electrode for implantation into muscle.

10. The system of claim 9 wherein said electrode is coupled to said vessel.

11. The system of claim 10 wherein said sensor is positionable upstream of said electrode.

12. The system of claim 1 wherein said output signal generated by said controller is based on an adaptive cancellation algorithm.

13. The system of claim 10 wherein the controller adjusts a delay time between the sensor signal and the output signal.
